(19) United States
(12) Patent Application Publication
(10) Pub. No.: US 2013/0261610 A1
(43) Pub. Date: Oct. 3, 2013

(54) SURGICAL INSTRUMENT HANDLE WITH CAM-ACTUATING SYSTEM

(71) Applicants: Matthew LaConte, O'Fallon, MO (US); Matthew Hanlon, O'Fallon, MO (US)

(72) Inventors: Matthew LaConte, O'Fallon, MO (US); Matthew Hanlon, O'Fallon, MO (US)

(73) Assignee: Synergetics, Inc., O'Fallon, MO (US)

(21) Appl. No.: 13/436,008

(22) Filed: Mar. 30, 2012

(51) Int. Cl. A61B 19/00; B23P 17/04

(52) U.S. Cl. 606/1; 29/428

(57) ABSTRACT

A surgical instrument handle having a cam-actuated system is disclosed. The surgical instrument handle includes a rotating member that defines a cam-actuating surface operatively engaged with a locking member, which is fixedly engaged to a reciprocating member for moving the reciprocating member between a retracted and extended positions. The cam-actuating surface of the rotating member is configured to permit the locking member to slide along the cam-actuating surface such that the tangential angle along the point of contact between the locking member and the cam-actuating surface is maintained at a predetermined angle.

### FIELD

This document relates generally to surgical instruments and in particular to surgical instrument handles having a cam-actuating system for operating a surgical instrument.

### SUMMARY

In an embodiment, a surgical instrument handle may include a handle body, a rotating member pivotally engaged to the handle body for rotation in first or second rotational directions wherein the rotating member defines a cam-actuating surface, a locking member configured to engage the cam-actuating surface of the rotating member, and a reciprocating member engaged to the locking member, wherein the rotational action of the rotating member in a first rotational direction causes lateral movement of the reciprocating member in a first longitudinal direction as the locking member travels along the cam-actuating surface of the rotating member and a rotational action of the rotating member in a second rotational direction causes lateral movement of the reciprocating member in a second longitudinal direction as the locking member travels along the cam-actuating surface of the rotating member.

In one embodiment, a surgical instrument handle may include a handle body, a rotating member pivotally engaged to the handle body, a locking member defining a cam-actuating surface and in sliding engagement with the rotating member, and a reciprocating member engaged to the locking member, wherein the rotational action of the rotating member in a first rotational direction causes lateral movement of the reciprocating member in a first longitudinal direction as the rotating member travels along the cam-actuating surface of the locking member and a rotational action of the rotating member in a second rotational direction causes lateral movement of the reciprocating member in a second longitudinal direction as the rotating member travels along the cam-actuating surface of the locking member.

In another embodiment, a method of manufacturing a surgical instrument handle may include:
forming a handle body;
forming a rotating member defining a cam-actuating surface and pivotally engaging the rotating member to the handle body;
engaging a locking member to the cam-actuating surface of the rotating member such that the locking member travels along the cam-actuating surface of the rotating member when the rotating member is rotated; and
coupling the locking member to a reciprocating member such that the travel of the locking member along the cam-actuating surface of the rotating member causes the reciprocating member to move in longitudinal direction.

In yet another embodiment, a method of manufacturing a surgical instrument handle may include:
forming a handle body;
pivotally engaging a rotating member to the handle body;
forming a locking member defining a cam-actuating surface and engaging the rotating member to the cam-actuating surface of the locking member; and
engaging the locking member to a reciprocating member;
wherein the travel of the rotating member along the cam-actuating surface of the locking member causes the reciprocating member to move in a longitudinal direction.

Additional objectives, advantages and novel features will be set forth in the description which follows or will become apparent to those skilled in the art upon examination of the drawings and detailed description which follows.

### BRIEF DESCRIPTION OF THE DRAWINGS

FIG. 1 is a side view of a surgical instrument handle;

FIG. 2 is a cross-sectional view of the surgical instrument handle taken along line 2-2 of FIG. 1;

FIG. 3 is an enlarged view showing a rotating member with a cam-actuating surface for the surgical instrument handle of FIG. 2 operatively engaged to a locking member;

FIG. 4 is an end view of the surgical instrument handle;

FIG. 5 is a top view of the surgical instrument handle;

FIG. 6 is an enlarged view of the surgical instrument handle of FIG. 3

FIG. 7 an exploded view of the surgical instrument handle illustrating the connection of the surgical instrument handle to an external connector through a cable;

FIGS. 8A-C are cross-sectional views of the surgical instrument handle showing a sequence of operation; and

FIG. 9 is an enlarged exploded view of the surgical instrument handle of FIG. 7;

FIG. 10 is a cross-sectional view of a tube and wire arrangement of the surgical instrument handle taken along line 10-10 of FIG. 2;

FIG. 11 is another cross-sectional view of the tube and wire arrangement of the surgical instrument handle taken along line 11-11 of FIG. 2;

FIG. 12 is a top view of another embodiment of the surgical instrument handle;

FIG. 13 is a cross-sectional view of the surgical instrument handle taken along line 13-13 of FIG. 12; and

FIG. 14 is an enlarged view of the surgical instrument handle of FIG. 13 showing a locking member with a cam-actuating surface operatively engaged to a pivotal rotating member.

Corresponding reference characters indicate corresponding elements among the view of the drawings. The headings used in the figures should not be interpreted to limit the scope of the claims.

### DETAILED DESCRIPTION

As described herein, a surgical instrument handle operates a surgical instrument using a cam-actuating system that translates rotational movement of one structural member into longitudinal movement of another structural member.

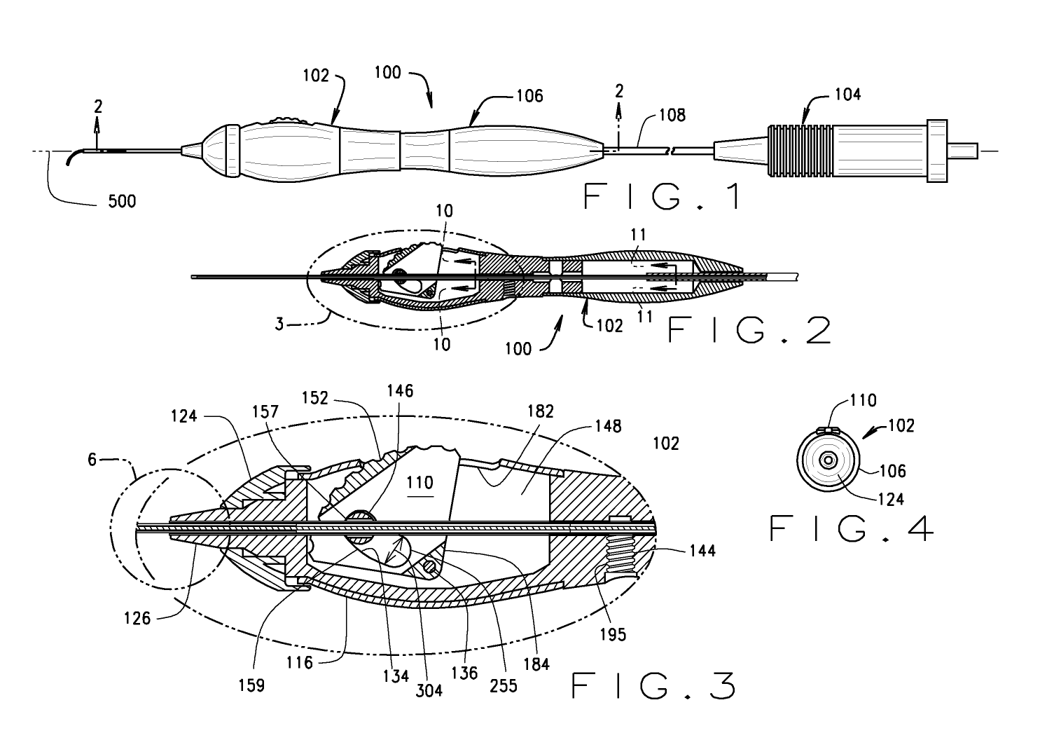

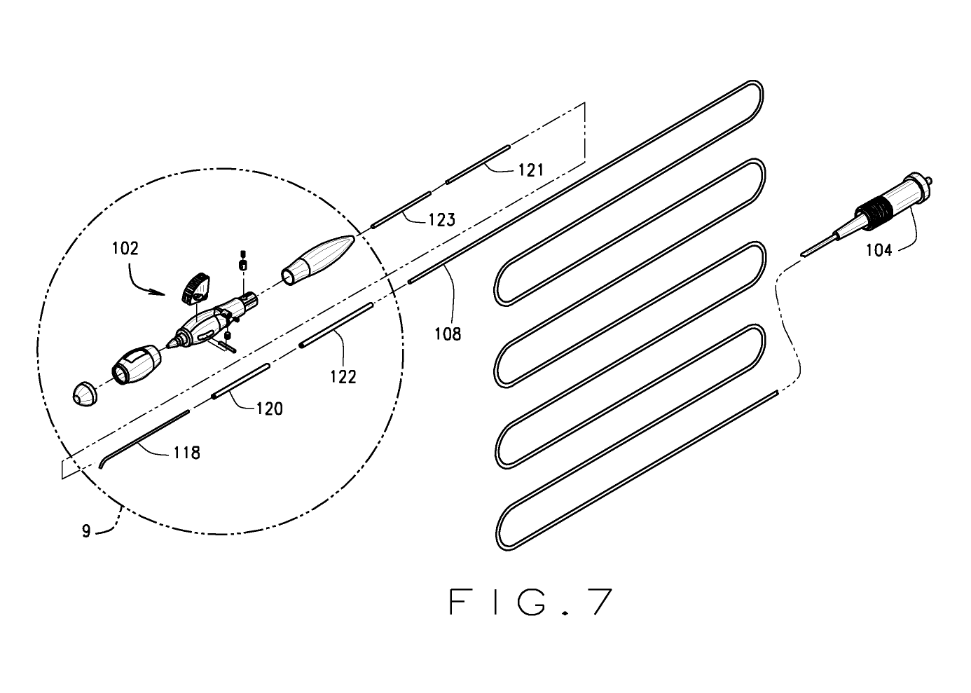

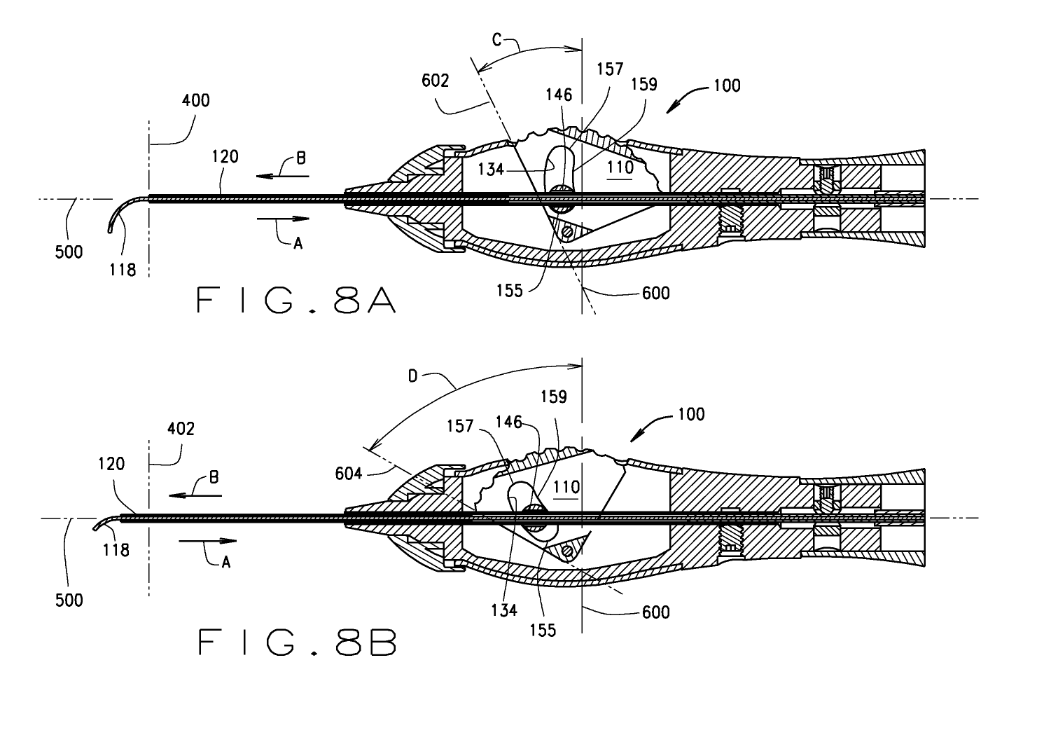

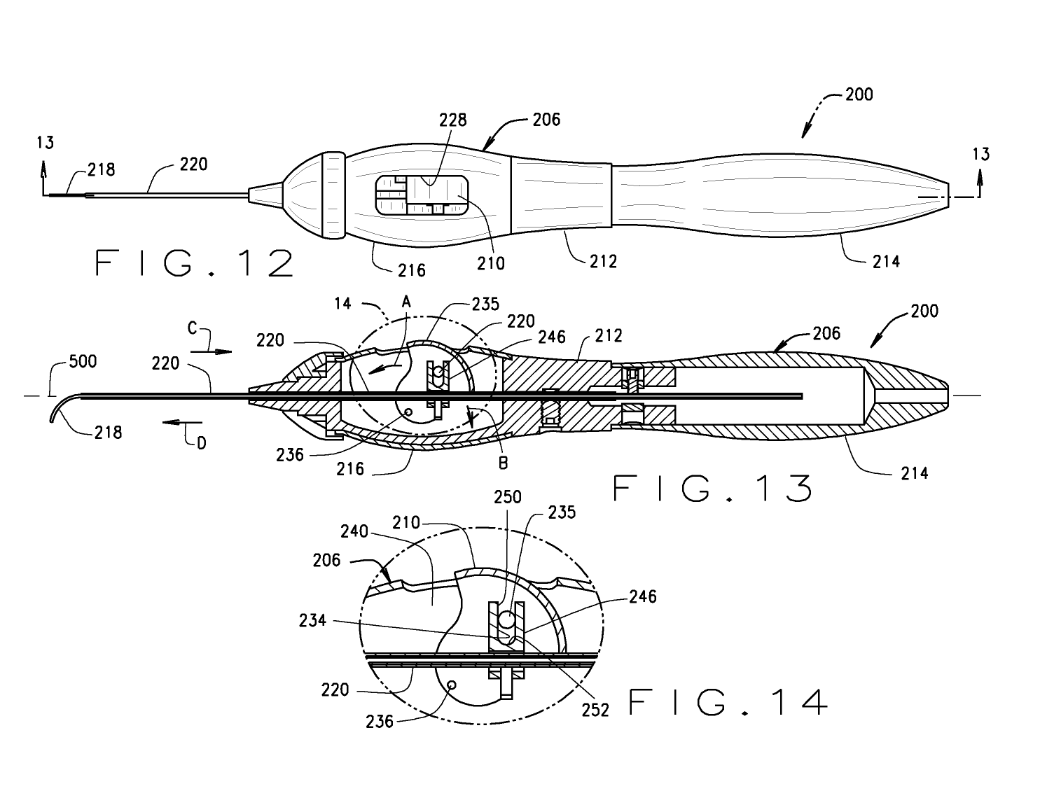

Referring to the drawings, various embodiments of a surgical instrument are illustrated and generally indicated as 100 and 200 in FIGS. 1-14. In one embodiment shown in FIG. 1, the surgical instrument 100 may be a laser probe apparatus having a surgical instrument handle 102 operatively connected through a cable 108, such as a fiber-optic cable, to a connector 104 for connecting the surgical instrument handle 102 to a source of power for operating the surgical instrument 100. In some embodiments, the surgical instrument handle 102 includes an elongated handle body 106 configured for one-handed operation by an individual in which rotation of a rotating member 110 (FIG. 3) that is pivotally mounted to the handle body 106 causes a retraction or extension action for operating the surgical instrument 100 as shall be described in greater detail below.

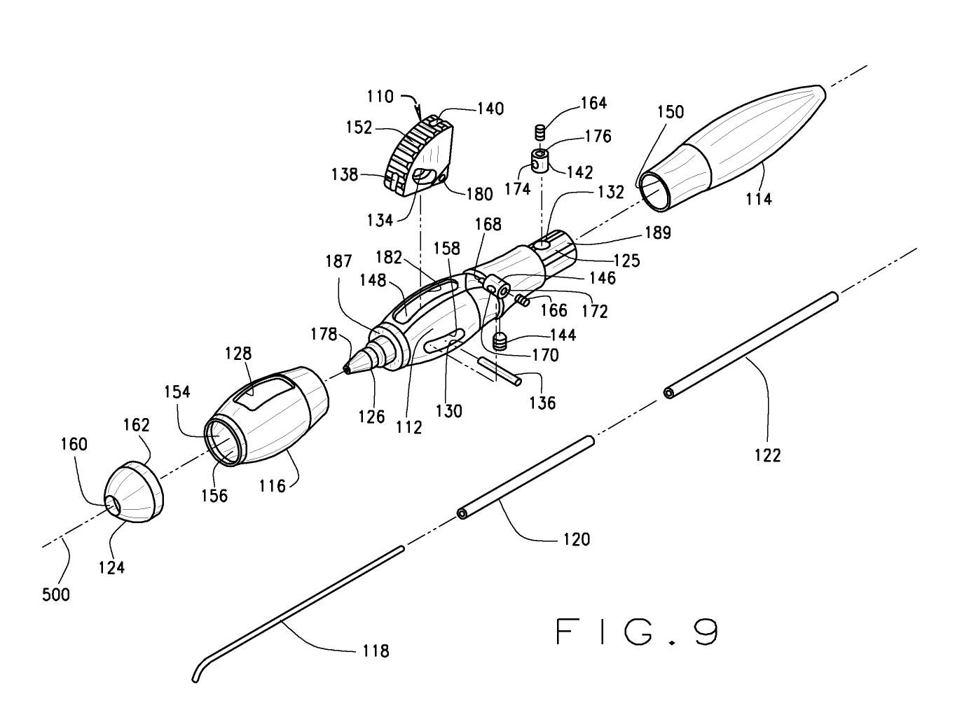

Referring to FIG. 9, one embodiment of the surgical instrument handle 102 includes an inner casing 112 having a proximal end 189 coupled to a rear casing 114 and a distal end 187 coupled to an outer casing 116. The outer casing 116 defines a longitudinally-extending top slot 128 that communicates with a chamber 148 defined within the inner casing 112 when the outer casing 116 is coupled to the inner casing 112. The outer casing 116 also defines a distal opening 154 configured to engage the proximal end 162 of a nose cone casing 124 having a nose cone opening 160 when the surgical instrument handle 102 is assembled. When the surgical instrument handle 102 is fully assembled, the inner casing 112, rear casing 114 and outer casing 116 collectively encase a hollow reciprocating member 120 in which a memory-shaped hollow tube 118 is coaxially disposed within the reciprocating member 120 that extends longitudinally through the handle body 106 substantially along longitudinal axis 500 (FIG. 1). In one embodiment, the memory-shaped hollow tube 118 is made from a memory-shaped material that may be bent and maintain the bent orientation. The reciprocating member 120 is operative to move between a fully extended position and a fully retracted position. In addition, the reciprocating member 120 is slidably engaged relative to the memory-shaped hollow tube 118 such that the reciprocating member 120 moves relative to the memory-shaped hollow tube 118, which is fixed in position. As shown, the reciprocating member 120 is extendable through a tip opening 178 of a nose cone fitting 126 attached to the inner casing 112 of the handle body 106.

As further shown, the inner casing 112 includes a rear portion 125 that defines a conduit 132 configured to receive a first locking member 142, which engages the memory-shaped hollow tube 118. In some embodiments, the memory-shaped hollow tube 118 may be nickel titanium, such as NITINOL™, or any other type of metallic material that may be bent and maintain a particular bent orientation. Specifically, the first locking member 142 defines an axial conduit 176 configured to receive a set screw 164 that communicates with a longitudinal conduit 174 configured to receive a portion of the memory-shaped hollow tube 118. When inserted into the axial conduit 174 the set screw 164 engages the memory-shaped hollow tube 118 such that the hollow tube 118 is fixedly secured in place relative to the inner casing 112 of the handle body 106.

Referring to FIGS. 3 and 9, when the handle body 106 is assembled a rotating member 110, such as a wheel or lever, is pivotally engaged to the inner casing 112 of the handle body 106 to permit an individual to actuate the surgical instrument handle 102. The rotating member 110 is pivotally engaged to the inner casing 112 through a pivot pin 136 inserted through the pin hole 180 formed through a bearing surface 184 of the rotating member 110 in a “slip fit” fashion that allows the rotating member 110 to pivot about an axis defined by the pin hole 180. In some embodiments, the inner casing 112 defines a side slot 158 configured to permit access to the rotating member 110 when engaging the locking member 146 to a curved slot 134 defined by the rotating member 110 during assembly of the surgical instrument handle 102. The bearing surface 184 provides a load bearing structure for the rotating member 110. The rotating member 110 defines a distal clearing notch 138 and a proximal clearing notch 140 that permit the rotating member 110 to clear the inner surface 182 of the chamber 148 when rotated in the most distal and proximal positions. The inner casing 112 further defines an aperture 130 configured to permit access to the rotating member 110 when inserting the pivot pin 136 through the pin hole 180.

In some embodiments, the rotating member 110 may define a kidney-shaped slot 134 that provides a cam-actuating surface configured to engage a locking member 146 which travels in sliding engagement along the kidney-shaped slot 134 when the rotating member 110 is rotated in either a clockwise direction or counter-clockwise direction. The kidney-shaped slot 134 may have a generally curved, non-linear configuration defining a first end 155, a second end 157 and a mid-point location 159 in which the width 304 of the slot 134 between any opposite lateral points remains substantially the same. As such, rotation of the rotating member 110 in either the clockwise or counter-clockwise rotational directions causes the locking member 146 to travel along the kidney-shaped slot 134 such that the tangential angle defined at each point of contact between the locking member 146 and the kidney-shaped slot 134 remains at about a 45 degree angle. In some embodiments, the tangential angle defined at the various points of contact between the locking member 146 and the kidney-shaped slot 134 may range between 35-50 degrees. In some embodiments, the rotating member 110 may be pivoted in a range of 90 degrees in either the clockwise and counter-clockwise directions. The rotating member 110 may further include a gripping surface 152 formed along the top peripheral portion of the rotating member 110, which defines a plurality of protrusions configured to engage the individual's thumb when rotating the rotating member 110 as shall be discussed in greater detail below.

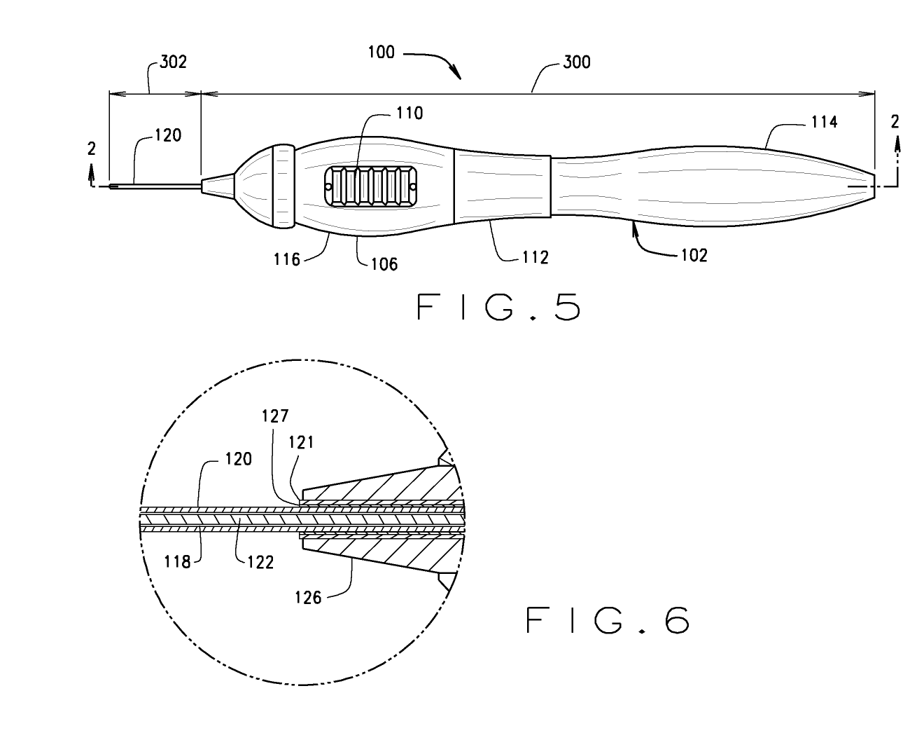

Referring to FIGS. 6, 7, 9 and 10, in some embodiments the distal portion of a fiber optic cable 122 may be coaxially disposed within the memory-shaped hollow tube 118 such that the orientation of the free end of the fiber optic cable 122 is set by the particular orientation of the memory-shaped hollow tube 118. In one arrangement, the memory-shaped hollow tube 118 may be coaxially disposed within the reciprocating member 120, which is capable of a sliding longitudinal movement relative to the memory-shaped hollow tube 118 in which the memory-shaped hollow tube 118 is fixed in position by the first locking member 142 relative to the reciprocating member 120. The hollow reciprocating member 120 may be coaxially disposed within a bearing tube 121 along longitudinal axis 500. In one aspect, the reciprocating member 120 may be engaged to the bearing tube 121, which is fixedly coupled to the handle body 106. As such, movement of the bearing tube 121 causes concurrent longitudinal movement of the reciprocating member 120 along longitudinal axis 500. In one embodiment, the reciprocating member 120 may be 20-gauge in size, although other gauges may be used in the construction of the reciprocating member 120. In an alternative embodiment, a bushing 127 (FIG. 6) may be coaxially disposed within the bearing tube 121 and surround the reciprocating member 120 in order to accommodate different coaxial arrangements of the hollow reciprocating member 120, bearing tube 121, and a memory-shaped hollow tube 118.

As shown in FIG. 3, the handle body 106 defines an axial conduit 195 in communication with a portion of the bearing tube 121 that extends longitudinally through the axial conduit 195. The axial conduit 195 is configured for threaded engagement with a tension-setting screw 144 that applies tension against the bearing tube 121, which allows the user to set the desired level of tension felt when manipulating the rotating member 110.

Referring to FIGS. 3, 7 and 9, the bearing tube 121 is configured to be inserted into a longitudinal conduit 170 defined by the second locking member 146. The second locking member 146 also defines an axial conduit 172 configured to receive a set screw 166 adapted to engage the bearing tube 121. In assembly, the second locking member 146 is in sliding engagement with the cam-actuating surface of the kidney-shaped slot 134 such that clockwise or counter-clockwise rotation of the rotating member 110 in a pivoting or rotating motion causes the second locking member 146 to travel along the cam-actuated surface of the kidney-shaped slot 134 in a cam action. The travel of the second locking member 146 along the cam-actuating surface of the kidney-shaped slot 134 translates the pivoting or rotational movement of the rotating member 110 into longitudinal movement of the bearing tube 121, which is fixedly attached to the reciprocating member 120, thereby driving the reciprocating member 120 in either the proximal or distal directions when the rotating member 110 is manipulated in a clockwise or counter-clockwise direction, respectively. In one embodiment, manipulation of the rotating member 110 in a clockwise direction causes the reciprocating member 120 to be extended outwardly from the handle body 106 in a distal direction relative to the memory-shaped hollow tube 118, thereby causing the memory-shaped hollow tube 118 to be fully disposed within the reciprocating member 120. Conversely, movement of the rotating member 110 in a counter-clockwise direction causes the reciprocating member 120 to be retracted inwardly into the handle body 106 in a proximal direction relative to the memory-shaped hollow tube 118, thereby causing the memory-shaped hollow tube 118 to be exposed. In other embodiments, the memory-shaped hollow tube 118 may move while the reciprocating member remains fixed in position relative to the surgical instrument handle 102.

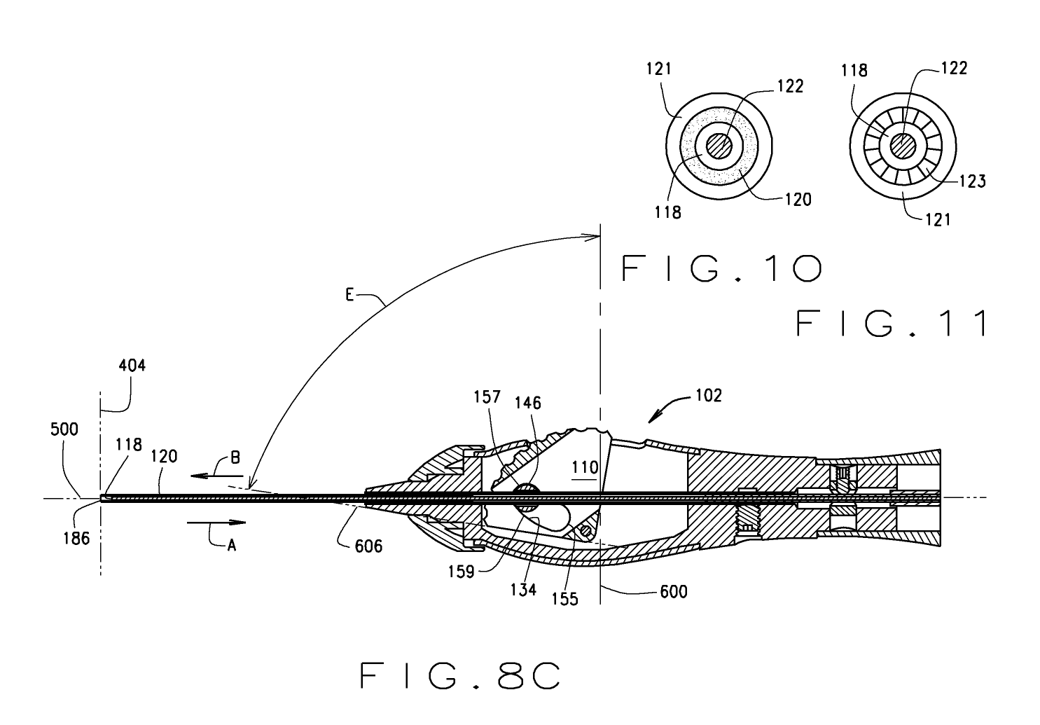

FIGS. 8A-8C show the sequence of the second locking member 146 in sliding engagement along the cam-actuating surface defined by the kidney-shaped slot 134 as the rotating member 110 is rotated as described above. In FIG. 8A, the reciprocating member 120 is shown in a fully retracted position such that the memory-shaped hollow tube 118 extends outwardly from the reciprocating member 120. In this position, the second locking member 146 is located at the first end 155 of the kidney-shaped slot 134 and the reciprocating member 120 is located at a first position 400. In the fully retracted position, the rotating member 110 is positioned along a plane 602, which forms an angle B with longitudinal plane 600. In FIG. 8B, the reciprocating member 120 is shown in a mid-retraction position such that less of the memory-shaped hollow tube 118 extends outwardly from the reciprocating member 120 than in the fully retracted position of the reciprocating member 120 as the rotating member 110 is rotated in a counter-clockwise direction. In this position, the second locking member 146 is located at the mid location 159 of the kidney-shaped slot 134 such that the reciprocating member 120 extends to a second position 402. In the mid retraction position, the rotating member 110 is positioned along a plane 604, which forms an angle C with longitudinal plane 600. In FIG. 8C, the reciprocating member 120 is shown in a fully extended position such that the memory-shaped hollow tube 118 is fully disposed within the reciprocating member 120 as the rotating member 110 has been further rotated in the counter-clockwise direction. In this position, the second locking member 146 is positioned at the second end 157 of the kidney-shaped slot 134 and the reciprocating member 120 extends to a third position 404. In the fully extended position, the rotating member 110 is positioned along a plane 606, which forms an angle D with longitudinal plane 600.

Conversely, when the rotating member 110 is rotated in the clockwise direction, the reciprocating member 120 may be moved from the fully extended position of FIG. 8C when the memory-shaped hollow tube 118 is fully disposed within the reciprocating member 120 to the fully retracted position of FIG. 8A when the memory-shaped hollow tube 118 extends fully outward from the reciprocating member 120.

Referring to FIGS. 3 and 11, in some embodiments a back tube 123 may be coaxially disposed within a portion of the bearing tube 121 proximal or adjacent to the reciprocating member 120. The back tube 123 may be capable of a sliding engagement with the bearing tube 121, which extends substantially the longitudinal length of the handle body 106. A portion of the memory-shaped hollow tube 118, proximal or adjacent to the reciprocating member 120 may be coaxially disposed within the back tube 123 and surround the fiber optic cable 122. However, in other embodiments

Referring to FIG. 5, in one embodiment the handle body 106 may have a length 300 of 105 mm and the reciprocating member 120 may extend a length 302 of 32 mm from the handle body 106 when in the fully extended position. However, in other embodiments the lengths 300 and 302 may vary in dimension.

Referring to FIGS. 12, 13 and 14, another embodiment of the surgical instrument, designated 200, is illustrated. The surgical instrument 200 includes a surgical instrument handle 202 having a handle body 206 that has an identical or substantially similar configuration to handle body 106 in which the handle body 206 includes an inner casing 212 coupled to an outer casing 216 and a rear casing 214. The outer casing 216 defines a longitudinal slot 228 having a rotating member 210, such as a wheel or lever, which extends partially outward from the longitudinal slot 228. The rotating member 210 is pivotally engaged to the handle body 206 through a pivot pin 236 that allows the rotating member 210 to pivot or rotate in either a clockwise direction or counter-clockwise direction within a chamber 240 defined by the inner casing 212. The rotating member 210 may include a rod 235 that extends laterally outward and is in sliding engagement with a cam-actuating surface 234 of a locking member 246, which is fixedly engaged to a reciprocating member 220. As such, rotation of the rotating member 210 in either the clockwise or counter-clockwise direction causes the rod 235 to travel along the cam-actuating surface 234 of the locking member 246 and drive the reciprocating member 220 in either the distal or proximal directions C and D along longitudinal axis 500.

In some embodiments, the cam-actuating surface 234 of the locking member 246 may have a U-shaped configuration that permits the rod 235 to travel between an open end 250 and a closed end 252 defined by the cam-actuating surface 234. Travel of the rod 235 between the open and closed ends 250, 252 causes the reciprocating member 220 to move in either the distal and proximal longitudinal directions C and D as described above.

In some embodiments, the surgical instrument handle 102, 202 may be adapted for use with any type of surgical instrument 100, 200 in which the travel of a first structural member along the cam-actuating surface defined by a second structural member drives a third structural member to move in a longitudinal direction for effecting an operation of the surgical instrument 100, 200. For example, the surgical instrument 100, 200 may be a laser probe disposed within a reciprocating member such that the reciprocating member can be retracted or extended relative to the laser probe using the motion imparted by the cam-actuating surface of a second structural member to retract or extend the reciprocating member relative to the laser probe. In other embodiments, travel of a first structural member along the cam-actuating surface of a second structural member may cause a third structural member to remotely deploy the surgical instrument 100, for example a stent (not shown), such that the reciprocating member is retracted by the travel of the first structural element along the cam-actuated surface that drives the reciprocating member to retract and deploy the stent. In some embodiments, the interaction of a first structural element with the cam-actuating surface defined by a second structural element may produce a mechanical action in a third structural element for operating surgical forceps or scissors in a cutting action. In some embodiments, the travel of a first structural element along the cam-actuating surface of a second structural element may produce a lateral action, a sliding action, a levering action, a cutting action, and/or a reciprocating action in a third structural element operatively engaged to the second structural element.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A surgical instrument handle comprising:
a handle body;
a rotating member pivotally engaged to the handle body for rotation in first or second rotational directions, wherein the rotating member defines a cam-actuating surface;
a locking member configured to engage the cam-actuating surface of the rotating member; and
a reciprocating member engaged to the locking member, wherein a rotational action of the rotating member in a first rotational direction causes lateral movement of the reciprocating member in a first longitudinal direction as the locking member travels along the cam-actuating surface of the rotating member and a rotational action of the rotating member in a second rotational direction causes lateral movement of the reciprocating member in a second longitudinal direction as the locking member travels along the cam-actuating surface of the rotating member.

2. The handle of claim 1, wherein the cam-actuating surface of the rotating member is configured to maintain a tangential angle at the point of contact between the locking member and the cam-actuating surface of the rotating member of between 35-50 degrees.

3. The handle of claim 1, wherein the locking member is configured to engage the reciprocating member such that the relative motion between the locking member and the cam-actuating surface of the rotating member causes the rotational movement of the rotating member to be translated to lateral movement of the reciprocating member.

4. The handle of claim 1, further comprising:
a first hollow tube disposed within the reciprocating member, wherein the first hollow tube is made from a memory-shaped material; and
a first wire coaxially disposed within the first hollow tube, wherein the first wire is an optical fiber.

5. The handle of claim 4, further comprising:
a second hollow tube having the first hollow tube coaxially disposed therein.

6. The handle of claim 6, further comprising:
a means for applying a degree of tension to the second hollow tube.

7. The handle of claim 1, wherein the rotating member defines a conduit configured to receive a pivot pin for permitting the rotating member to rotate in the first rotational direction or rotate in the second rotational direction.

8. The handle of claim 1, wherein the rotating member defines a gripping surface comprising a plurality of protrusions.

9. The handle of claim 1, wherein the rotating member defines a distal clearing notch and a proximal clearing notch for allowing the rotating member to be moved to a distal-most position or a proximal-most position, respectively, without interference from the handle body.

10. The handle of claim 1, further comprising:
a second locking member secured to the first wire for fixing the first wire in a stationary position such that the reciprocating member moves relative to the stationary position of the first wire.

11. The handle of claim 1, wherein the reciprocating member is a hollow tubular member.

12. The handle of claim 1, wherein the cam-actuating surface is defined along an interior surface of the rotating member, wherein the interior surface defines a slot having a first end and a second end, wherein the cam-actuating surface is configured to produce a cam action by the locking member as the locking member moves between the first end and the second end of the cam-actuating surface that translates rotational movement of the rotating member to longitudinal movement of the reciprocating member.

13. The handle of claim 1, wherein the locking member defines a longitudinal conduit which is configured to operatively engage the reciprocating member.

14. The handle of claim 1, wherein the handle body comprises an inner casing coupled to an outer casing at a distal end of the inner casing and a rear casing coupled to a proximal end of the inner casing.

15. The handle of claim 1, wherein the rotating member is rotated in either a clockwise or counterclockwise direction for causing retraction or extension of the reciprocating member.

16. The handle of claim 1, wherein the rotating member is a wheel or a lever.

17. A surgical instrument handle comprising:
a handle body;
a rotating member pivotally engaged to the handle body;
a locking member defining a cam-actuating surface and in sliding engagement with the rotating member; and
a reciprocating member engaged to the locking member, wherein a rotational action of the rotating member in a first rotational direction causes lateral movement of the reciprocating member in a first longitudinal direction as the rotating member travels along the cam-actuating surface of the locking member and a rotational action of the rotating member in a second rotational direction causes lateral movement of the reciprocating member in a second longitudinal direction as the rotating member travels along the cam-actuating surface of the locking member.

18. The handle of claim 17, wherein the cam-actuating surface of the locking member defines an open end and a closed end.

19. The handle of claim 18, wherein the rotating member includes a rod configured to engage the cam-actuating surface of the locking member and travel between the open end and the closed end of the cam-actuating surface when the rotating member is rotated in a pivoting action.

20. A method of manufacturing a surgical instrument handle comprising:
forming a handle body;
forming a rotating member defining a cam-actuating surface and pivotally engaging the rotating member to the handle body;
engaging a locking member to the cam-actuating surface of the rotating member such that the locking member travels along the cam-actuating surface of the rotating member when the rotating member is rotated; and
coupling the locking member to a reciprocating member such that the travel of the locking member along the cam-actuating surface of the rotating member causes the reciprocating member to move in longitudinal direction.

21. The method of claim 20, wherein the cam-actuating surface of the rotating member is configured to maintain a tangential angle at the point of contact between the locking member and the cam-actuating surface of the rotating member of between 35-50 degrees.

22. The method of claim 20, wherein the cam-actuating surface of the rotating member forms an interior curved slot.

23. A method of manufacturing a surgical instrument handle comprising:
forming a handle body;
pivotally engaging a rotating member to the handle body;
forming a locking member defining a cam-actuating surface and engaging the rotating member to the cam-actuating surface of the locking member; and
engaging the locking member to a reciprocating member;
wherein the travel of the rotating member along the cam-actuating surface of the locking member causes the reciprocating member to move in a longitudinal direction.

24. The method of claim 23, wherein the rotating member includes an engaging member configured to travel along the cam-actuating surface of the locking member.
